(19) United States
(12) Patent Application Publication
(10) Pub. No.: US 2009/0326621 A1
(43) Pub. Date: Dec. 31, 2009

(54) Surgical delivery devices and methods

(76) Inventor: Rizk El-Galley, Birmingham, AL (US)

Correspondence Address: Ballard Spahr LLP, SUITE 1000, 999 PEACHTREE STREET, ATLANTA, GA, 30309-3915, US

(21) Appl. No.: 11/920,911

(22) PCT Filed: May 24, 2006
(86) PCT No.: PCT/US2006/020231

§ 371 Date: Aug. 28, 2009

Related U.S. Application Data
(60) Provisional application No. 60/684,047, filed on May 24, 2005.

(51) Int. Cl. A61F 7/12

(52) U.S. Cl. 607/105

(57) ABSTRACT

A surgical device and method for using the same for delivering a cooled solution to a subject can include a reservoir configured to contain a cooled and a tubular probe in communication with the reservoir. In operation, a portion of the tubular probe is inserted through a laparoscopic port and into a desired location within the subject so that the solution in the cooled state can be selectively dispensed from the end of the tubular probe and into the desired location within the subject.

This application claims priority to U.S. Provisional Application No. 60/684,041, filed on May 24, 2005. The aforementioned application is herein incorporated by this reference in its entirety.

### BACKGROUND

Laparoscopic surgery is increasingly common. The principle of laparoscopic surgery is to perform a surgical procedure with small keyhole incisions. Usually, two or three such keyhole incisions are made in the abdomen for insertion of a telescopic video camera, laparoscopic instruments and electrosurgical devices.

The advances of laparoscopic surgery over the past decade have revolutionized the approaches to renal surgery. Most surgical centers consider laparoscopic surgery as a first choice for renal tumors that are confined to the kidney and do not have tumor thrombus extending beyond the renal vein. Similarly, laparoscopic donor nephrectomy has become the standard in most centers. The results of laparoscopic donor nephrectomy compared to open donor nephrectomy are well known in the art and show equivalent graft function in both procedures. Laparoscopic donors benefit from the advantages of laparoscopic surgery which include less postoperative pain, early recovery, and smaller scars.

During partial or full nephrectomy, blood supply to the kidney is temporarily stopped. It is ideal to cool the kidney during interruption of the blood supply to avoid kidney damage. During open abdominal surgery, this cooling is accomplished by applying ice to the kidney. However, during laproscopic partial nephrectomy, the size of the laproscopic port holes are too small to allow for application of ice to the kidney. Devices and methods are needed for cooling of organs and/or tissues during laparoscopic and non-laparoscopic surgical procedures.

### SUMMARY

Provided herein are surgical devices and methods for delivering a cooled solution to a subject. An exemplary embodiment of a surgical device of the present invention comprises a reservoir configure to contain a cooled solution and a tubular probe in communication with the reservoir. In operation, a portion of the tubular probe is inserted through a laparoscopic port and into a desired location within the subject so that the solution in the cooled state can be selectively dispensed from the end of the tubular probe and into the desired location within the subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a schematic diagram showing an exemplary surgical device.

FIG. 2 is a schematic diagram showing an exemplary surgical device.

FIG. 3 is a schematic diagram showing an exemplary surgical device.

FIG. 4 is a schematic diagram showing an exemplary surgical device.

FIG. 5 is a schematic diagram showing an exemplary surgical device.

FIG. 6 is a schematic diagram showing an exemplary surgical device.

FIG. 7 is a schematic diagram showing an exemplary surgical device.

FIG. 8 is a schematic diagram showing an exemplary surgical device.

FIG. 9 is a schematic diagram showing an exemplary surgical device.

FIG. 10 A, B, and C are schematic diagrams showing an exemplary surgical devices.

FIG. 11 is a schematic diagram showing an exemplary surgical device.

FIG. 12 is a schematic diagram showing an exemplary surgical device.

FIG. 13 is a schematic diagram showing an exemplary surgical device.

### DETAILED DESCRIPTION OF THE INVENTION

The present invention may be understood more readily by reference to the following detailed description and the Examples included therein and to the Figures and their previous and following description.

Before the present articles, devices, and/or methods are disclosed and described, it is to be understood that this invention is not limited to specific synthetic methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a gas inlet port” includes two or more such ports, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally the tubular probe is stainless steel” means that the tubular probe may or may not be stainless steel and that the description includes tubular probes that comprise stainless steel and tubular probes that comprise materials other than stainless steel.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

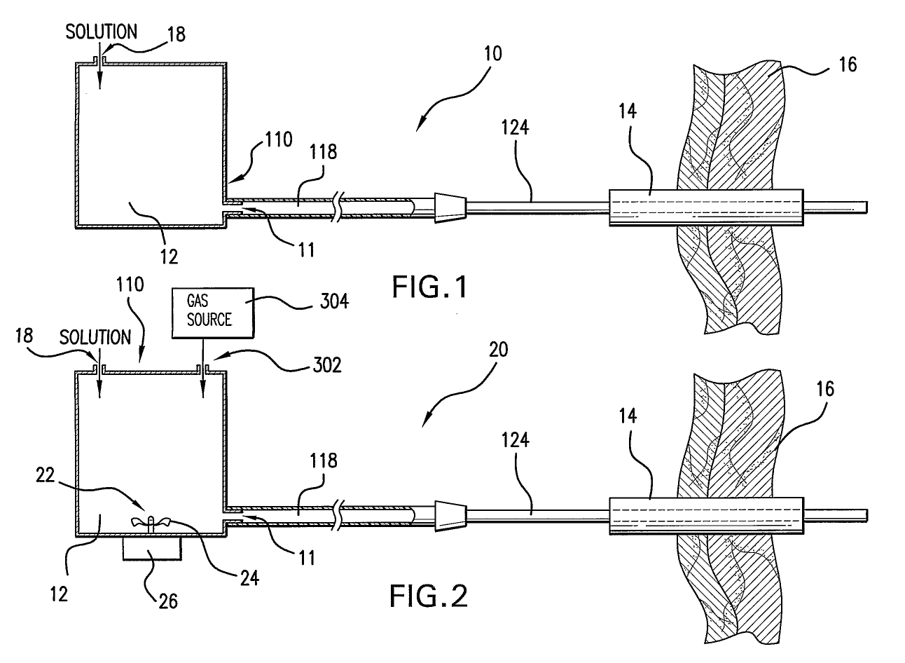

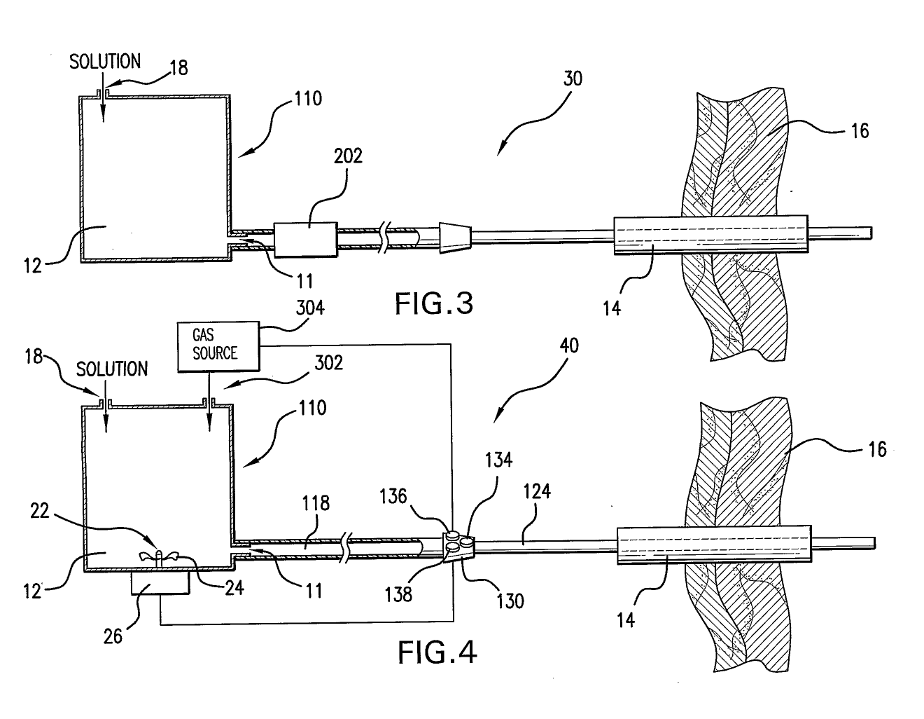

FIG. 1 illustrates an exemplary surgical device 10 for dispensing a cooled solution during surgical procedures on a subject. The surgical device 10 can comprise a reservoir 110 defining an interior cavity 12. The reservoir is configured to maintain a solution disposed therein the interior cavity in a cooled state. The device can further comprise a tubular probe 124 having a first end and an opposed second end. The first end of the tubular probe 124 can be in selective fluid communication with the interior cavity 12 of the reservoir 110 such that the solution in the cooled state can be selectively dispensed from the second end of the tubular probe into a subject. The tubular probe can be configured for insertion through a conventional laparoscopic port or a trocar 14. The trocar or laparoscopic port 14 can be located across a subject's body wall 16 from outside of the subject and into a body cavity of the subject.

FIG. 2 illustrates an exemplary surgical device 20 that can comprise a mixer having a motorized mixing member. The mixer can thus comprise a motor 26 and a mixing member 22. At least a portion of the mixing member 22 can be disposed within the interior cavity 12 of the reservoir 110. In operation, the mixing member can be moved by the motor to move cooled solution located within the interior cavity. The cooled solution can be pre-cooled and introduced into the interior cavity through an inlet 18. Ice and cooled or non-cooled solution can also be introduced to into the interior cavity 110. In one aspect, the mixing member 22 can crush the ice and mix ice and solution until a desired consistency of fluid or solution within the interior cavity is achieved. In a further aspect, the mixing member 22 can comprise a blade 24 for contacting and crushing or cutting the ice in the interior cavity. Fluid or solution can also be introduced into the interior cavity and cooled to produce a cooled solution or slush of desired consistency. Any combination of ice, fluid, and/or solution can be cooled and/or maintained in a cooled state by the surgical device.

The term “maintained” it is not intended to be limited to the maintenance of a solution within the interior cavity at a steady state temperature, and fluctuations in temperature are also intended to be included by the term “maintained.” Thus, solution is considered to be maintained in a cooled state when it is held in the interior cavity such that it can be dispensed at a desired range of temperatures for use in a surgical procedure that is being performed. One skilled in the art can readily determine a desired range of temperatures for a dispensed solution. In one aspect, the solution is maintained in the device at the desired range of temperatures such that the solution is dispensed from the tubular probe 124 at a temperature within the desired range of temperatures.

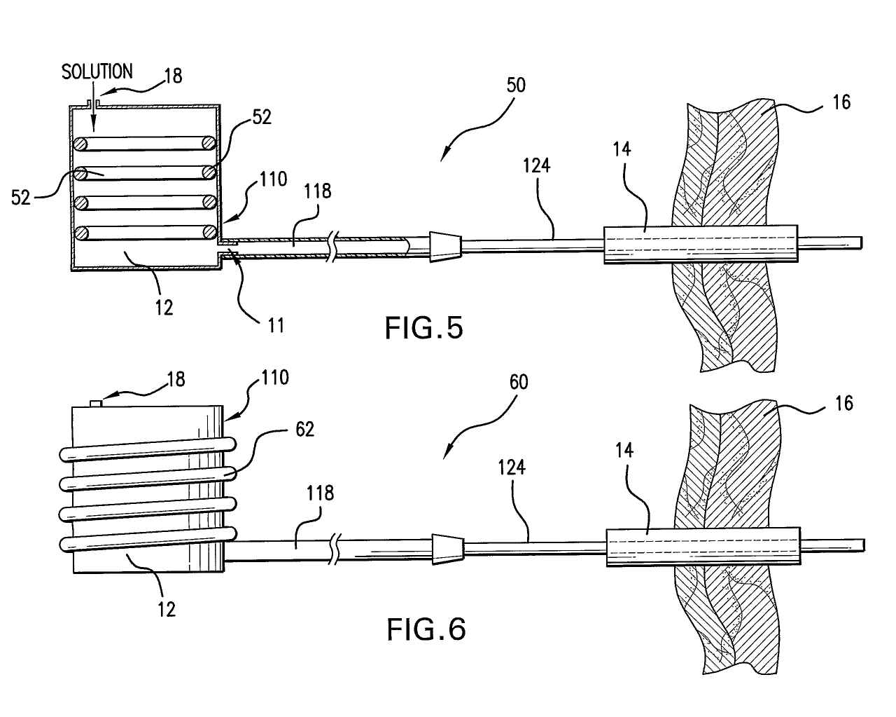

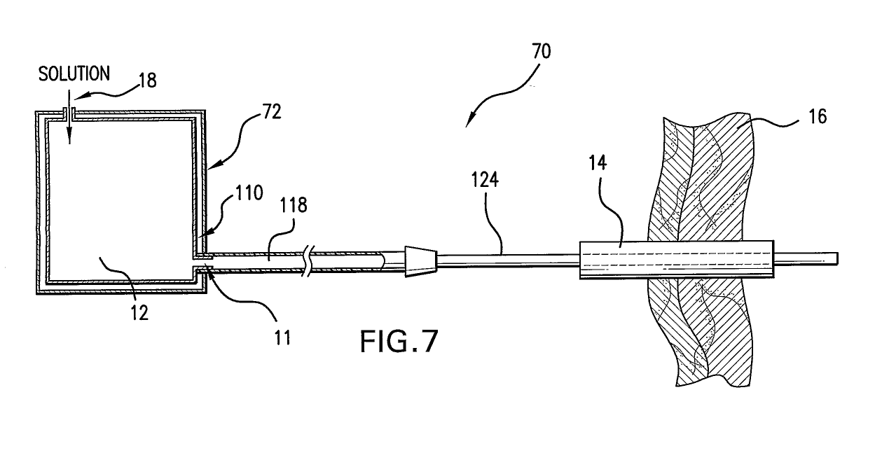

In exemplary aspects, the surgical devices can actively cool and or insulate the solution located therein the interior cavity. For example, as shown in FIGS. 5, 6 and 7, the surgical device can comprise a cooling member, as shown by 52, 62, and 72 respectively, for cooling or maintaining the solution in a cooled state. For example, the surgical device can comprise a cooling pipe configured for maintaining the solution in its cooled state. In one aspect, at least a portion of the cooling pipe can be positioned within the interior cavity of the reservoir as shown in FIG. 5. Optionally, the cooling pipe can comprise a coil. In a further aspect, at least a portion of the cooling pipe can be positioned about a portion of an external surface of the reservoir as shown in FIG. 6. In this aspect, the cooling pipe can exemplarily comprise a coil that is wrapped about and contacts a portion of the external surface of the reservoir. In another aspect, the cooling member can be embedded or at least partially embedded in the reservoir. Optionally, the cooling member can also comprise a sleeve 72 that is located about at least a portion of the reservoir's exterior surface.

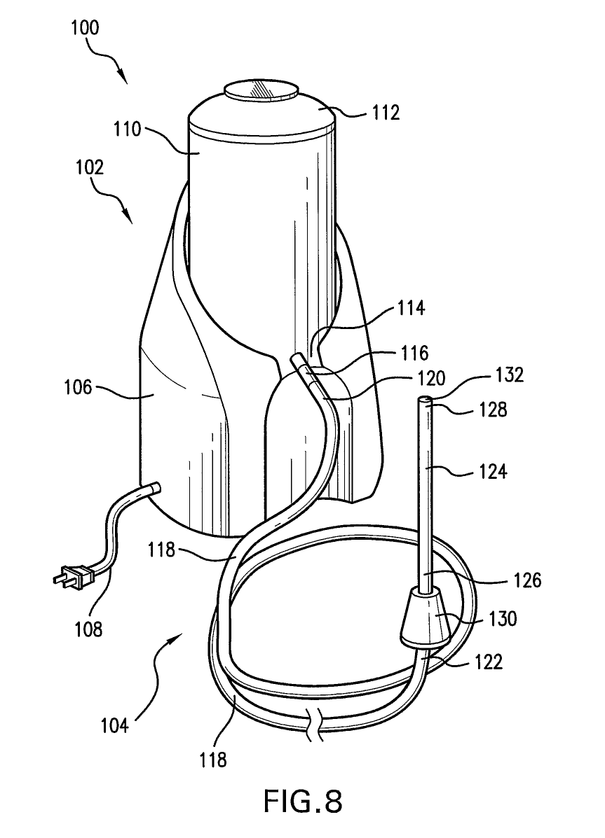

In a further aspect of the present invention and as shown in FIG. 8, the reservoir 110 can have a top portion 112 and a bottom portion 114. The bottom portion of the reservoir can be configured to seat on a base portion 106 of the device. The motor 26 of the mixer can be disposed therein an interior portion of the base member 106. When seated, the mixing member 22 can be in operative connection with the motor 26 of the mixer.

In another aspect, the device can further comprise a delivery portion 104. The delivery portion is configured to deliver cooled solution from the interior cavity 12 to the subject. In one aspect, the delivery portion can comprise the tubular probe 124. The delivery portion can further comprise a conduit 118 that extends between a distal end and an opposed proximal end. In one aspect, the distal end of the conduit 118 is connected to an outlet port 11 of the reservoir and the proximal end of the conduit is connected to the first end of the tubular probe. In this configuration, the conduit receives cooled solution from the interior cavity through an outlet port on the reservoir. Thus, the distal end of the conduit is in fluid communication with the interior cavity of the reservoir. In one aspect, the tubular probe can comprise an elongated shaft that has a first end, a spaced second end and defines a lumen therethrough. The first end of the tubular probe can be in fluid communication with the proximal end of the conduit. In another aspect, at least one opening 132 is located at the second end of the tubular probe. In a further aspect, at least a portion of the tubular probe, including the second end, is configured to be positionable laparoscopically within a subject such that the device can be used in methods of laparoscopic surgery and in methods of open surgery.

Further provided herein is a method of cooling tissue within a subject comprising inserting at least a portion of the tubular probe 124 into position within the subject on the selected tissue or in proximity to the tissue. The method further comprises producing an ice and fluid tissue cooling mixture and administering the cooling mixture through the lumen of the tubular probe 124. The administered cooling mixture contacts the select tissue or a location within the subject that is in proximity to the tissue and acts to reduce the temperature of the tissue. Thus, cooled solution can be dispensed through the tubular probe 124 and into a subject.

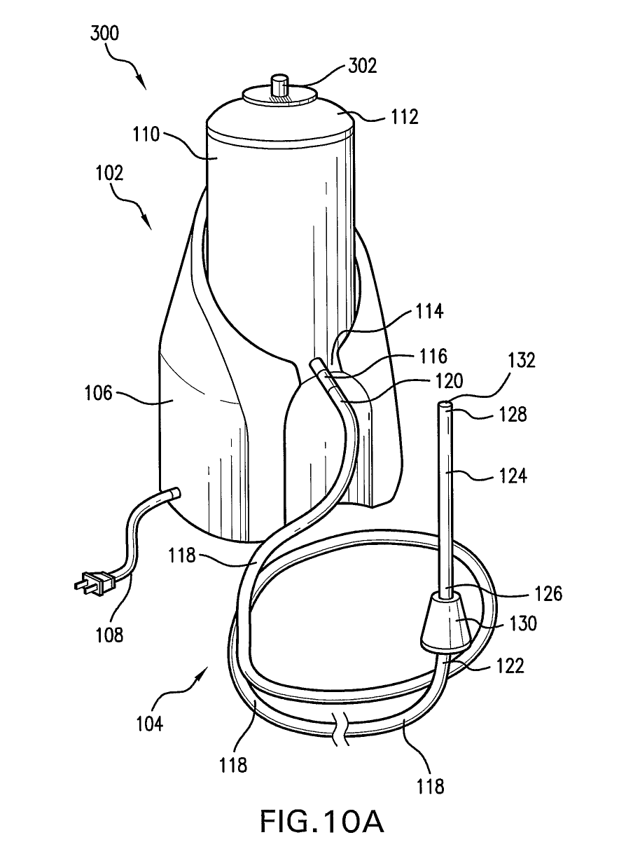

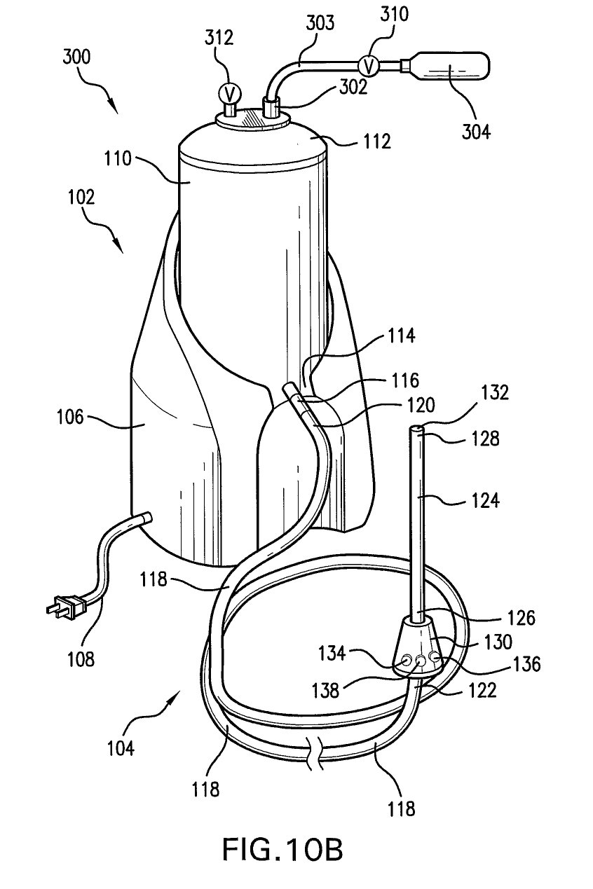

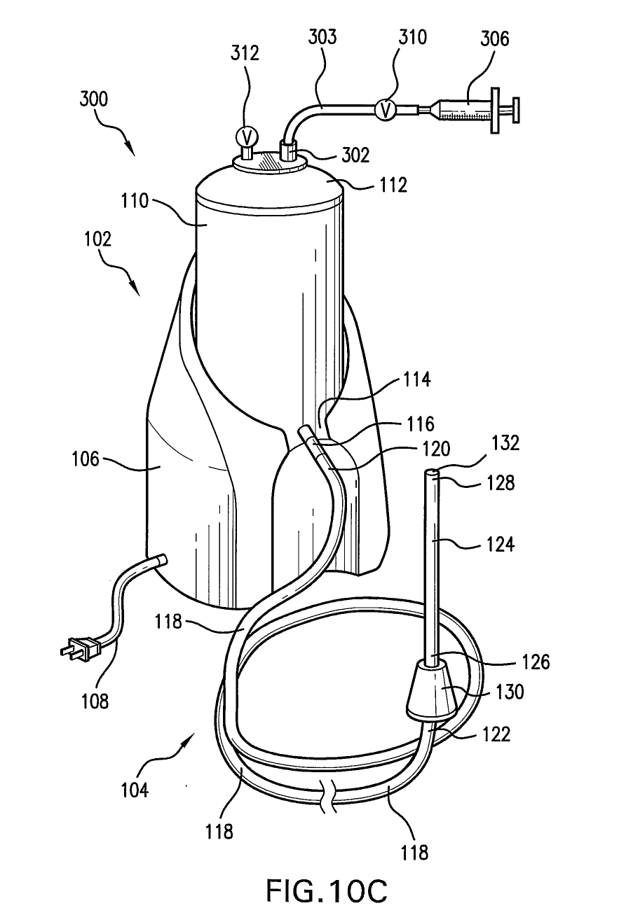

In a further aspect of the invention, the cooled solution can be propelled through the tubular probe for disposition within the subject. Gas can be introduced into the interior cavity 12 of the reservoir to can increase the gas pressure within the interior cavity. The increased pressure can be used to propel the cooled solution out of the probe and into desired disposition within the subject. As shown in FIG. 2, the reservoir can comprise a gas inlet port 302 configured for the introduction of gas into the interior cavity of the reservoir. Optionally, the reservoir 110 can further comprise a gas outlet port 312 configured for release of gas from the interior cavity of the reservoir as shown in FIGS. 10B and 10C. In one exemplary aspect, the gas outlet port can comprise a one-way pressure release valve.

In a further aspect. the surgical devices can further comprise a source of pressurized gas 304 in fluid communication with the gas inlet port 302 as shown in FIGS. 2, 4, 10B and 10C. As one will appreciate, the source of pressurized gas can be used to introduce gas into the interior cavity for pressurizing the interior cavity. In this aspect, the surgical device can further comprise a regulating mechanism 310 configured to prevent or reduce the flow of gas from the source 304 to the interior cavity as shown in FIGS. 10B and 10C. It is contemplated that the regulating mechanism 310 can be selectively operable by a user of the surgical device to allow, to prevent, or to reduce the flow of gas from the source to the interior cavity. In exemplary aspects, the source of gas can be pressurized and can be selected from a group consisting of: a pump, a syringe 306, and a pressurized gas container.

Embodiments of an exemplary surgical device 100 are shown in FIGS. 8 through 13. The illustrated exemplary surgical devices can comprise a reservoir and/or mixer portion 102 and delivery portion 104. The reservoir and/or mixing portion can comprise a base member 106 and an ice/fluid reservoir 110. The reservoir can have a top portion 112 and a bottom portion 114 that can be seated on the base member 106 such that a motor located therein the base member can drive a crushing and/or mixing member 22 located within the interior cavity 12 of the reservoir 110.

The motor of the base member 106 can be powered by electricity through a power cord 108 or a battery. It is contemplated that the motor can be powered by any conventional means, as would be clear to one skilled in the art.

Optionally, the motor 26 located therein the base member 106 causes rotation of a crushing/mixing member 22 in a clockwise or counter-clockwise direction. The resultant rotation of the crushing/mixing member can act to crush ice and to move the resulting fluid/ice mixture in a clockwise or counterclockwise direction throughout the reservoir 110. In one aspect, the crushing/mixing member can comprise a blade 24 for contacting and cutting or crushing the ice. Optionally, the base member 106 can further comprise a motor 26 for causing the rotation of an extruder type screw mechanism. Such an extruder mechanism is known in the art and is typically located in the bottom portion of the interior space of the reservoir, but it can also be located in a valve 116 or along other portions of the delivery portion. The extruder type screw mechanism can crush, mix and move the mixed and crushed mixture into and through the delivery portion of the device.

The ice/fluid reservoir can be used to hold or maintain ice, saline, water, Ringer's solution, any other fluid used in surgical procedures to irrigate surgical sites, or combinations thereof. Ice can be mixed with one or more fluids to be crushed and mixed to form a tissue cooling mixture or cooled solution for laparoscopic administration to tissue or an organ of a subject. The reservoir 110 can vary in size, as would be clear to one skilled in the art. By non-limiting example, the interior cavity of the reservoir can be configured to hold between about one to about two gallons of cooled solution. However, reservoirs having interior cavities less than one gallon or greater than two gallons can also be used depending on the amount of ice and fluid or ice/fluid mixture desired for a given surgical procedure, or depending on other factors such as the delivery of ice and fluid from the reservoir to the delivery portion 104.

Optionally, the base member 106 comprises a multi-speed blender type base. The motor can be operated at one or more speeds. For example, two speeds, with one speed being a lower speed, and one speed being a higher speed can be used. In an exemplary operation, at the lower speed, the motor drives the crushing/mixing mechanism used to crush and move fluid throughout the ice/fluid reservoir at a lower speed. At the higher speed, the motor moves the crushing/mixing mechanism at a higher speed to crush the ice and move the fluid at a higher rate throughout the ice/fluid reservoir. In one aspect, the base member 106 can comprise a rheostat for the control of voltage to the motor. Thus, the exemplified base member 106 can operate very much like a conventional blender base that is used to blend ice and fluids. If an extruder type screw mixing and crushing mechanism is used, the motor can be configured to turn the screw in either and/or both a clockwise or counterclockwise direction and can turn the screw at a variety of speeds depending on the desired mixture flow characteristics.

The base member 106 can be made of a variety of materials, such as, metals, rubbers, plastics or polymers, or otherwise appropriate materials, which can be selected by one skilled in the art. Optionally, the base member is autoclavable or can be otherwise be sterilized. The ice/fluid reservoir 110 can also be made of plastic, metal, rubber, polymers, or combinations thereof, and can optionally be autoclavable or otherwise sterilized.

In various aspects, the ice/fluid reservoir 110 can be used to hold the fluids and/or ice that is crushed, blended and/or mixed to form the cooled fluid mixture that can be subsequently delivered into the subject through the delivery portion 104 for cooling of tissue or an organ during laparoscopic surgery. Thus, fluid and ice added to the reservoir can be used to make an ice “slush” or tissue cooling “mixture” of ice and fluid that can be delivered into and through the delivery system 104, for example, and not meant to be limiting, by gravitational flow, through pressurized flow, through centrifugal force, through a pump mechanism 202 located along the delivery portion, through a screw type extruder mechanism (not shown), and the like.

In one aspect, the ice and fluid, and any desired medications or compositions can be added into the ice/fluid reservoir 110 through an opening or inlet 18 in the reservoir. In another aspect, the top portion 112 can be removable as is known in the art and is similar to the jug of a typical kitchen blender. One skilled in the art will also appreciate that ice and/or fluid can also be added to the reservoir through any other type of inlet or opening in the reservoir or through retrograde flow through the delivery portion 104.

The desired proportions of ice and fluid added to the ice/fluid reservoir can be determined by one of skill in the art and may depend on factors such as the type of surgery being performed, the length of time an organ will need to be cooled, the desired temperature to which an organ is to be cooled, and other factors that can be readily determined by history, physical examination and the pathological condition of the subject.

As noted above, the ice and/or fluid located in the reservoir 110 can be blended or crushed by the mixing member. As it is crushed, the mixture can be swirled about in a clockwise or counter-clockwise direction in the reservoir, depending on the direction that the motorized crushing implement or mixing member is rotating. The rotation of the ice and fluid as it is crushed and/or blended can cause the mixture to be forced toward the periphery of the container walls.

In one aspect, centrifugal force of the fluid or cooled solution moving within the interior of the reservoir can cause the fluid or solution to be forced through a valve 116 and into the delivery conduit 118. The valve can also be considered part of the delivery portion 104, and in particular, can comprise the distal end of a delivery conduit 118. Optionally, the valve can be a one-way or a two-way valve. In one aspect, a one-way valve can allow passage of the mixture from the reservoir into the delivery portion 104, but without allowing retrograde flow from the delivery member back into the reservoir's interior cavity. In an alternative aspect, a two-way valve can allow antegrade and retrograde flow across the valve, which allows flow of the mixture from the reservoir's interior cavity into the delivery member and from the delivery member back into the reservoir's interior cavity. The valve can be in fluid communication with the lumen of the delivery conduit as described below. It is contemplated that an extruder type screw mixing mechanism can be integrated into the valve or into the first end of the delivery conduit.

In a further aspect, the delivery portion 104 can comprise a delivery conduit 118 having a distal end 120 and an opposed proximal end 122 that is configured for the flow of the fluid/ice mixture from the reservoir. In one aspect, the distal end can be in fluid communication with the inner cavity of the reservoir by operative connection to an outlet defined on the reservoir surface. Optionally, if a valve 114 is used, the valve can be placed in fluid communication with the distal end of the delivery conduit and in fluid communication with the interior cavity defined by the reservoir. As described above, however, the valve 114 can also be an integral part of the delivery conduit 118, such that the first end of the delivery conduit is in fluid communication with the interior conduit defined by the inner surface of the reservoir.

In another aspect, the delivery portion can further comprise a tubular probe 124. The tubular probe has a first end, a spaced or opposed second end and defines a lumen therethrough. In this aspect, the first end of the tubular probe can be in fluid communication with the distal end of the delivery conduit and is thus placed in fluid communication reservoir's interior cavity. In a further aspect, at least one opening can be located at the second end of the tubular probe or along an intermediate portion of the tubular probe. In this aspect, at least a portion of the tubular probe, including the second end, is configured to be positionable laparoscopically within the subject. In another aspect, the tubular probe can have at least one opening that is configured to allow fluid to flow through the delivery portion 104 and to exit the surgical device to contact a desired organ or structure, or proximate location thereto within a subject.

It is contemplated that the length of the delivery conduit can vary. Optionally, the delivery conduit 118 is approximately one yard long. In another example, the delivery conduit is approximately two yards long. Any length, however, could be used including lengths around and between one and two yards, less than one yard, or greater than two yards. The diameter of the delivery conduit 118, as specified by the luminal diameter of the conduit can also vary. Optionally, the luminal diameter is approximately 5.0 mm, 10.0 mm or 12.0 mm. The tubular probe 124 can also vary in diameter. In another aspect, the diameter of the lumen of the tubular probe can be, for example, approximately 5.0, 10.0 or 12.0 millimeters. Nominally the surface of the tubular probe is typically of negligible thickness and at least a portion of the tubular probe 124 is sized and shaped to be inserted through a conventional laparoscopic port or trocar having about a 5.0 mm, 10.0 mm, or 12.0 mm diameter. Thus, the tubular probe 124 can be inserted into a subject through a laparoscopic port or trocar during laparoscopic surgery.

The delivery conduit 118 or tubular probe 124 can be made of a variety of materials, including plastics, rubbers, or polymers as would be clear to one skilled in the art. The delivery conduit can be flexible and can be made of common surgical or medical tubing. The delivery conduit 118 and/or tubular probe 124 can include any medical-surgical tubing. The delivery tube or tubular probe can be made of materials common in the art suitable for use as medical or surgical tubing as would be clear to the skilled artesian. Such exemplary tube/conduit material includes tubing used for medical procedures such as, but not limited to, intravenous tubing, airways, catheters, shunts, drains, dialysis tubing and parenteral feeding tubing. Optionally, the delivery conduit 118 and/or tubular probe 124 can be made of a biologically inert material, which can be of a semi-rigid plastic. Flexible polyvinyl chloride plastics, such as those used in medical tubing and catheters, are suitable. Sources for this material can be found by reference to Rubin, “Handbook of Plastic Materials and Technology” (1990).

The tubular probe 124 can also be made of a variety of materials, including plastics, rubbers, metals or other polymers as would be clear to one skilled in the art. Optionally, the tubular probe is made of stainless steel. Optionally, the tubular probe 124 is substantially rigid and is made of a metal such as stainless steel or of a hard plastic or other rigid or semi-rigid material. As one will appreciate, the tubular probe can also vary in length and is optionally approximately 6-20 inches long, although tubular probes shorter than 6 inches or longer than 20 inches can also be used. Optionally, the tubular probe is about 15 inches long. The delivery conduit 118, regulating mechanism 130, and tubular probe 124 can be autoclavable or disposable as would be clear to one skilled in the art.

In a further aspect of the invention and as shown in FIGS. 4 and 10B, the surgical devices can optionally comprise a regulating mechanism 130. In one aspect, the regulating mechanism can be located between the proximal end 122 of the delivery conduit and the first end 126 of the tubular probe. In another aspect, the regulating mechanism 130 can be used to selectively stop or allow fluid passage through the delivery system 104. Thus, it is contemplated that the regulating mechanism 130 can be any type of clamp, valve, or stop cock that can be used to selectively prevent or allow the flow of fluid through a tube. The regulating mechanism can also be located at any other position along the delivery portion 104 of the device.

In varying aspects, the regulating mechanism can be any mechanism capable of stopping flow through the valve 116, the delivery conduit 118, the tubular probe 124, or any other members of the delivery member 104. One example of a regulating mechanism is a stop cock. Other examples would be any mechanism capable of being opened or closed to allow fluid flow respectively. Specifically, such a mechanism can be, but is not limited to, a clamp or valve. In one aspect, the regulating mechanism can be manually controlled. For example, the surgeon or any other technician or medical professional, can manually open or close the regulating mechanism as desired. In another aspect, the regulating mechanism can be controlled by automatic switching means. For example, software can be programmed or a timer can be used to open or close the regulating mechanism at designated times. The timing of opening and closing can depend on the procedure to be performed, the extent of the subject's disease, the physician's preference or other variables as would be clear to one skilled in the art.

Operational controls allowing for an operator to selectively open and close any portion of the delivery portion can be located on the regulating mechanism 130. For example, a switch or button 134 can be located on the regulating mechanism 130. The switch or button 134 can be adjusted by the operator to allow, reduce, or prevent flow of fluid through any portion of the device by selectively obstructing flow through the conduit or probe. Additional operational controls (136 and 138) can similarly be located on the regulating mechanism. For example, a switch or button 136 that allows the user adjust the introduction of gas into the interior cavity can be located on the regulating mechanism. Moreover, a switch or button 138 that allows the user adjust the speed or use of the mixing member can be located on the regulating mechanism. In another aspect, operational control for some features can be combined. For example, one operational control can be used to regulate the introduction of gas into the interior cavity and the movement of the mixing member.

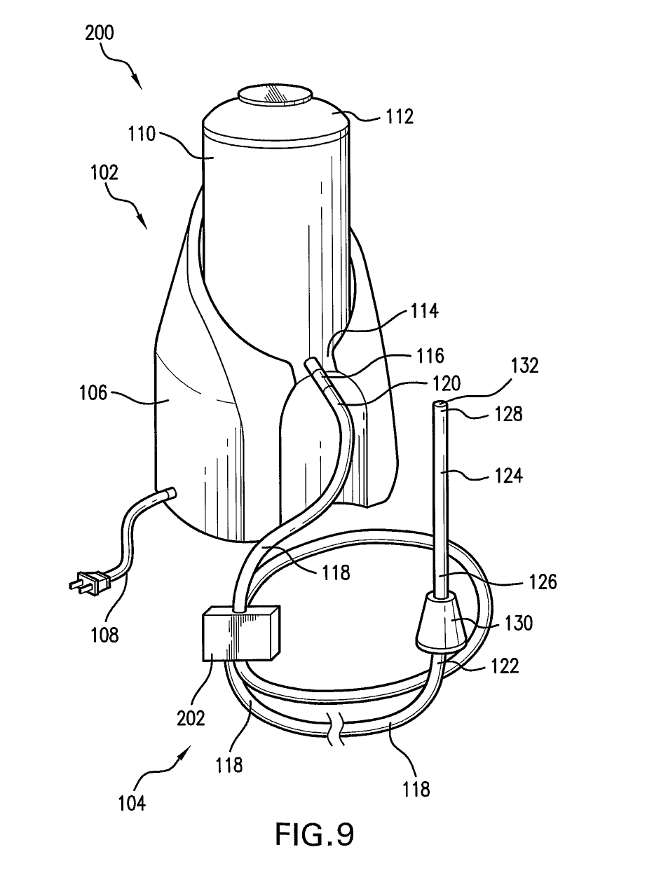

FIG. 9 shows a schematic diagram of an exemplary surgical delivery device with a fluid pumping mechanism 202. In one aspect, the pumping mechanism 202 can be any type of pump, and can be powered by electricity and/or battery or otherwise powered as would be clear to one skilled in the art. Optionally, the pump is a peristaltic pump in which the delivery conduit 118 is integrated into the pumping mechanism to force fluid or cooled solution through the lumen of the delivery conduit in a direction typically from the distal end of the conduit 120 towards the proximal end of the conduit 122. Thus, the pumping mechanism can be used to drive fluid or cooled solution along the delivery conduit, through the tubular probe 124, and out of the second end 128 of the tubular probe for delivery to a target of interest during laparoscopic surgery.

Peristaltic pumps typically use rotating rollers pressed against flexible tubing to create or enhance a pressurized flow. When a peristaltic pump is used, typically nothing but the tube 118 touches the fluid, eliminating the risk of the pump contaminating the fluid, or the fluid contaminating the pump. If a peristaltic pump is used, fluid can be drawn into the pump, trapped between two shoes (in a Bredel pump) or rollers (in a Watson-Marlow pump), and expelled from the pump along the delivery tube 118. Closure of the delivery conduit lumen, which can be squeezed between a shoe and the track, can give the pump its positive displacement action, preventing backflow and eliminating the need for check-valves when the pump is not running.

Other types of pumps 202 can also optionally be used include, but not limited to the following: positive displacement pumps, which use a mechanical force to push liquid through and out of the pump; hand pumps and foot pumps, which are manually operated pumps that can driven by hand or foot via a handle or lever; piston pumps and plunger pumps, which are reciprocating pumps using a plunger or piston to move the media through a cylindrical chamber; lobe pumps, which are positive displacement devices that use rotating lobes to direct flow; dosing pumps, which are low-volume fluid pumps with controllable discharge rates used to inject additives into the mixing or pumping system; metering pumps are precision pumps, which dispense an exact amount of material; screw pump, which are rotary, positive displacement pumps that have one or more screws to transfer fluids or materials along an axis; diaphragm pumps, which use a diaphragm that moves back and forth to transport liquids from one place to another; progressing cavity pumps, which are a type of rotary positive displacement pump designed to transfer fluid or media with suspended solids or slurries from the suction side of the pump to the discharge side of the pump; centrifugal pumps, which comprise a set of rotating vanes, enclosed within a housing or casing, and the like.

FIGS. 10 A, B and C are schematic diagrams showing exemplary surgical devices. FIG. 10A shows a device 300 having an inlet port 302 located on the top portion 112 of the ice/fluid reservoir 110. In one aspect, the inlet portion 302 can be located at other points on reservoir 110, however, and can be located at point above the fluid and/or ice mixture.

FIG. 10B shows an attached cartridge of pressurized gas to the inlet port 302. For example, a CO₂ cartridge can deliver pressurized gas through the inlet port 302. Although FIG. 10B, shows the pressurized gas source 304 in direct contact with the inlet 302, the gas source can be operatively connected to the inlet thorough a connecting mechanism such as an intermediate tube 303. Thus, gas can be introduced into the interior cavity by way of the inlet and an intermediate tube or gas flow line from the gas source. The cartridge of pressurized gas can be used to deliver gas through the inlet port 302 and into the reservoir 110 to pressurize the gas in the reservoir 110. Pressurizing the reservoir's interior cavity can be used to force the ice/fluid mixture from the reservoir's interior cavity into and through the delivery portion 104 of the device. The gas in the reservoir 110 can be pressurized to approximately 25 to 30 mm/Hg. Other pressures can also be achieved, however, and may be desired depending on the desired flow characteristics through the delivery system 104 and to the subject.

FIG. 10C shows another exemplary method of pressuring the gas in the reservoir 110 through the inlet port 302 using a common syringe 306. In this aspect, force applied to the plunger of the syringe 306 can be used to force gas into the reservoir 110 through the inlet port 302, causing a rise in the pressure within the reservoir 110. A pressure of approximately 25-30 mm/Hg can be achieved.

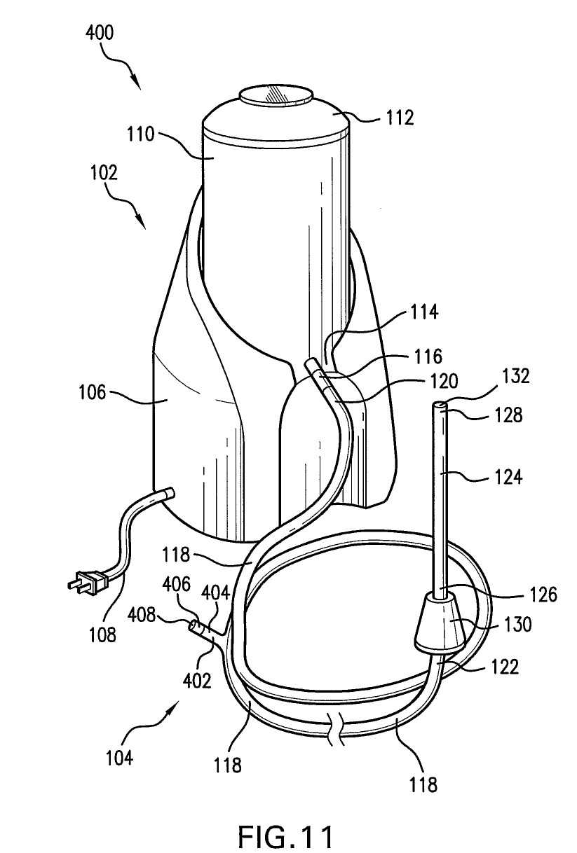

FIG. 11 is a schematic diagram showing an exemplary surgical device wherein the delivery conduit 118 comprises a branch 402 that is positioned along the delivery conduit 118. The branch 402 has a first-end 404, a spaced second end 406, and defines a lumen therethrough that is in fluid communication with the lumen of delivery conduit 118. The branch 402 can further comprises a needle injection port 408, located at the second end 406 into which a needle can be inserted to deliver fluid and/or gas from a syringe or similar mechanism into and through the lumen of the branch and into and through the lumen of the delivery conduit 118, and subsequently through the lumen of the tubular probe 124.

Fluid and/or gas injected through the branch can be used as a flush to relieve blockage along the delivery portion 104 of the device. Thus, although the branch is shown, spaced from the first end of the delivery conduit 118 and from the valve 116, it should be clear that the branch can be located anywhere along the delivery portion of the device or at the valve 116. In a further aspect, multiple branches can also be located at multiple locations such that flush can be delivered to any location of the delivery portion of the device 104.

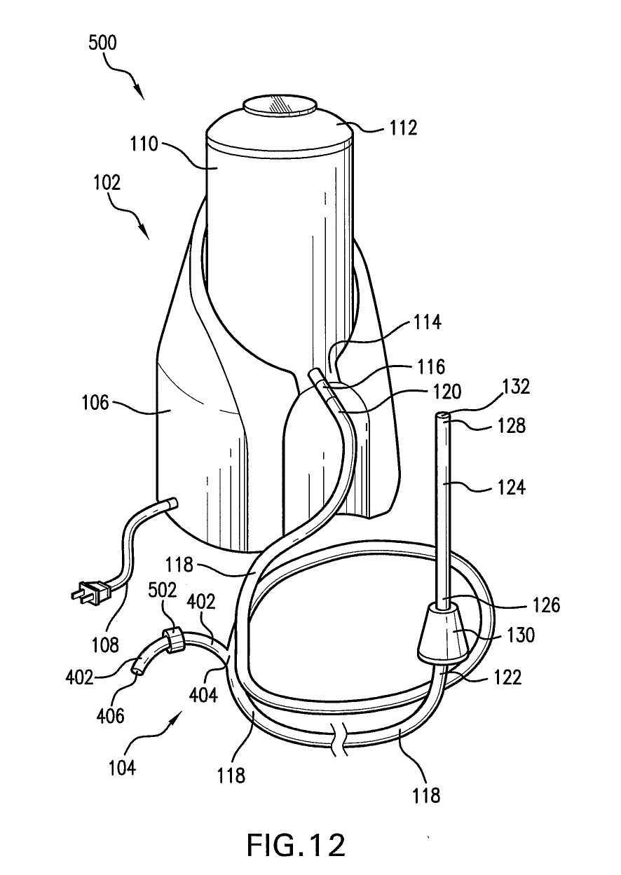

FIG. 12 is a schematic diagram showing an exemplary surgical device wherein the branch 402 has a clamp or valve 502 located therein. In this example, there may not be a needle injection port located at the second end of the branch. However, the second end of the branch can be adapted to receive fluid from any fluid source, such as the bag or container of fluid which can be delivered by gravitational force or pressure through the branch 402 and into the lumen of the delivery conduit 118, the lumen, the tubular probe, or at any point along delivery member or valve as described above. The clamp or valve 502 can selectively allow or prevent fluid flow into or through the branch.

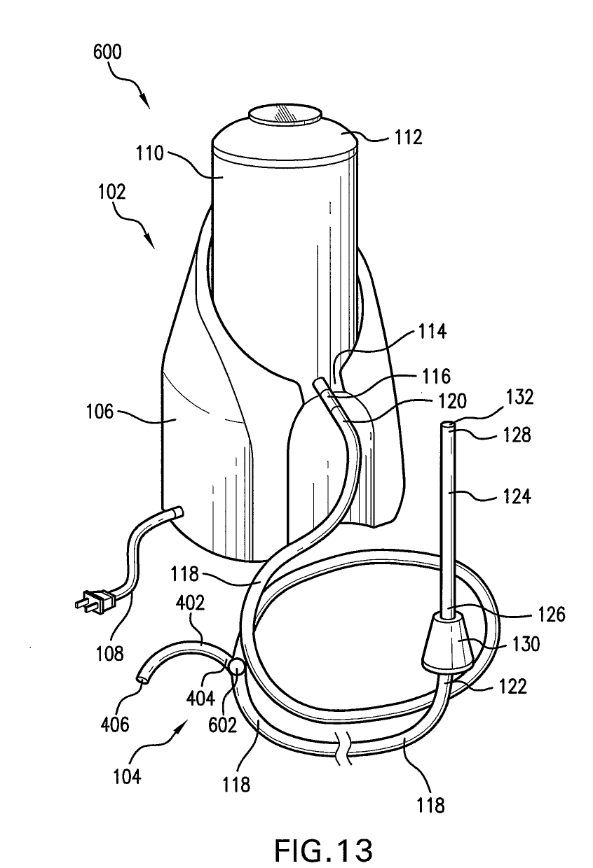

FIG. 13 is a schematic diagram showing a surgical device wherein a valve 602 is positioned to selectively allow fluid flow through a lumen of the delivery conduit 118 and/or through the branch 402. Thus, the valve 602 can be rotated or otherwise manipulated and can be, for example and not meant to be limiting, a one-way, two-way or three-way valve. Thus, the valve can be manipulated to allow fluid flow simultaneously through both delivery conduit 118 and the branch 402, individually through the branch 402, or individually through delivery conduit 118. It is contemplated that the valve and the branch can be located at any location along the delivery portion 104, however, including in the tubular probe, at the valve 116, or at multiple points there between. As described above, flush can be added at the second end 406 and can be selectively allowed to flow into the lumen of delivery portion to remove any blockage of the mixture flowing from the reservoir through delivery portion to the subject.

### EXAMPLES

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The surgical device can be used to induce tissue or organ hypothermia during a laparoscopic surgical procedure. Optionally, the device is used during laparoscopic partial nephrectomy, a procedure during which blood supply to the kidney is temporarily stopped. Once the blood supply is stopped, surgeons rapidly complete the surgical procedure to avoid damaging renal function under conditions of warm ischemia.

Renal hypothermia protects against potential renal ischemic injury and allows for a longer ischemic time. Using the described device and methods, renal hypothermia is achieved. For example, the renal parynchemal temperature can be reduced to about 30° C., 25° C., 20° C., 15° C., 10° C., 5° C. or less. Such temperatures can provide 2-3 hours or more of renal ischemia without permanent renal injury. As would be clear to one skilled in the art, however, the device and methods described herein are not limited to laparoscopic partial nephrectomy. The device and methods can also be used for other laparoscopic procedures such as, but not limited to, liver surgery and can also be used for non-laparoscopic surgical or medical procedures where the delivery of a cooling mixture or slush is desired. Also, the device and methods can be used for non-laparoscopic surgical procedures.

An ice/fluid mixture or slush is prepared by placing desired quantities of ice and fluid into the reservoir 110. Any suitable ratio of ice to fluid (ice:fluid) can be used, as can be determined by one skilled in the art. Typically, the ratio of fluid to ice is selected such that when the ice is crushed and mixed with the fluid, a slurry or slush is formed that can move into and through the delivery portion 104 of the device and can be dispensed within the subject. Optionally, only ice is added to the reservoir. Optionally, only fluid is added to the reservoir. If only fluid is added to the reservoir, it can be pre-cooled. The device can also be configured to cool any solution or fluid or ice/fluid combination introduced therein the interior cavity. The ice and/or fluid can be sterile, as can the any portion of the device. Moreover, the delivery device portions can be disposable. A variety of fluids can be used such as, for example and not meant to be limiting, lactated Ringer's solution, water, or other solutions commonly used for irrigation during surgery, and combinations thereof. Moreover, the frozen form of any of these fluids can be used as ice.

For laparoscopic kidney surgery, a retroperitoneal, a transperitoneal, or a hand assisted approach can be used as described in Bishoff and Kavoussi, Atlas of Laparoscopic Retroperitoneal Surgery, W.B. Saunders Co., Chapter 103, 3645-3681 (2000), which is incorporated by reference for the surgical methods taught therein. Moreover, modifications of such traditional approaches and other approaches can be used as determined by the surgeon performing the procedure. For each approach, the subject can be positioned as appropriate. For example, for the retroperitoneal approach, the subject can be placed in the standard flank position. The disclosed devices and methods can also be used in open surgery without using a laparoscopic approach and can also be used for open surgery of organs including, but not limited to the kidney.

For any approach used, one or more incisions are made and trocars are inserted thorough the incisions. Before trocar placement, the abdomen can be insufflated using a Veress needle. Visualizing means can be placed through one of the ports. The visualization port can be between 10 mm and 12 mm in diameter. Additional trocars can therefore be placed under direct visualization if desired. Each additional trocar is typically 5 mm, 10 mm or 12 mm in diameter. A variety of surgical tools or devices, for example, but not limited to a retractor, grasper, or forceps can be inserted through a trocar or port site. Optionally, four incisions, laparoscopic port, or trocar sites are used. The renal artery and/or vein can be occluded using a surgical clamp, such as a bulldog clamp.

The ice/fluid mixture or slush located in the reservoir 110 is delivered to the subject. Such a delivery can include the deposition of a desired volume of the mixture on or around the subject's kidney, or into a mechanism, such as a bag, designed to accumulate the mixture at the surgical site. The mixture can be delivered to the subject before, during and/or after the renal artery/vein is clamped. Thus, the mixture can be delivered at any time throughout a surgical procedure. Delivery intervals can be determined by the surgeon or another medical professional and may depend on the surgery's duration, the subject's condition, complications, or on any other medically or surgically relevant consideration that could be determined by on skilled in the art.

The ice/fluid mixture is typically delivered to the subject at the desired site through at least one opening located at or near the second end 128 of the tubular probe 124. At least a portion of the tubular probe, including the second end 128, can be inserted into the subject through one of the trocar or laparoscopic port sites. Moreover, if an additional source of cooling mixture is desired, at least a portion of an additional tubular probe or probes can be inserted through one or more additional port. In this aspect, any additional tubular probe can be in fluid communication with an additional reservoir 110 through additional delivery conduits, or can be attached to the same reservoir 110 as the first tubular probe.

With the tubular probe in its appropriate position to deliver the mixture to the kidney or surgical location, the mixture is moved through the tubular probe lumen and expelled from the tubular probe opening. As noted above, the mixture can be moved along the tubular probe by gravity, by the centrifugal force of the mixture within the reservoir 110, by pressurizing the gas within the reservoir as described above, by a extruder type screw mechanism, by a pump mechanism, or by a combination thereof. These forces typically cause the mixture to enter the delivery system 104 though the valve 116 which is in fluid communication with the distal end 120 of the delivery conduit 118. The fluid or cooled solution moves along the lumen of the delivery conduit, towards the proximal spaced end 122 of the delivery conduit. The lumen of the delivery conduit can be in fluid communication with the lumen of the tubular probe 124, or a regulatory mechanism 130 can be interposed between the lumen of the delivery conduit and the tubular probe. The regulatory mechanism 130 can be used to selectively prevent, allow, or regulate the volume or speed of flow from the lumen of the delivery tube into the lumen of the shaft member.

In operation, the mixture delivered on, around, or in proximity to the kidney or surgical site causes renal hypothermia. The temperature of the kidney can be monitored by inserting a thermometer into the kidney parenchyma at the surgical site. By monitoring the temperature of the kidney at the surgical site, the surgeon can determine whether the desired level of renal hypothermia has been achieved, or whether additional ice/fluid mixture or slush should be delivered within the subject. The temperature detected by the thermometer can be used as a feedback to regulate the additional flow of the mixture into the subject or the stoppage or reduction of flow into the subject. Such regulation can be accomplished by automated switching means and/or feedback control means.

As shown above, the delivery conduit, valve 114 or tubular probe can also be in fluid communication with a branch 402. The branch can be used to flush the valve, delivery conduit, or tubular probe. Thus, a separate source of fluid can be administered through the delivery system using the branch 402. The separate source of fluid can be used as a flush if the delivery system becomes clogged or blocked. Moreover, any number of medications can be added through the branch line or into the reservoir 110, if desired. For example and not meant to be limiting, antibiotics, anesthetics, vaso-constricting agents, chemotherapeutics, and the like, can be added.

The mixture administered to the subject, or fluid located within the subject resulting from the administration of the fluid or cooled solution, can be removed from the subject by suction back through the delivery portion or members thereof or by a separate laparoscopic suction mechanism. Optionally, the fluid removed from the subject by suction back through the delivery portion can be directed into a separate container. Thus, a three-way valve, which can be manually or automatically switched, as described above, can be used to direct the retrograde fluid flow into the separate container. If, after suctioning of the fluid into the container, additional cooling mixture is to be administered to the subject, the three-way valve can be switched allowing the cooling mixture to again flow form the reservoir through the delivery portion and into the subject. The fluid can also be drained from the subject using a separate drain or similar mechanism as would be clear to one skilled in the art.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A surgical device, comprising:
a reservoir defining an interior cavity, wherein the reservoir is configured to maintain a solution disposed therein the interior cavity in a cooled state; and
a tubular probe having a first end and an opposed second end, wherein the tubular probe is configured for insertion through a laparoscopic port, and wherein the first end of the tubular probe is in selective fluid communication with the interior cavity of the reservoir such that the solution in the cooled state can be selectively dispensed from the second end of the tubular probe into a subject.

2. The surgical device of claim 1, further comprising a conduit extending therebetween a distal end and an opposed proximal end, wherein the distal end of the conduit is connected to an outlet port of the reservoir, and wherein the proximal end of the conduit is connected to the first end of the tubular probe.

3. The surgical device of claim 1, wherein the reservoir further comprises a gas inlet port configured for the introduction of gas into the interior cavity of the reservoir.

4. The surgical device of claim 3, wherein the reservoir further comprises a gas outlet port configured for release of gas from the interior cavity of the reservoir.

5. The surgical device of claim 4, wherein the gas outlet port comprises a one-way pressure release valve.

6. The surgical device of claim 3, further comprising a source of pressurized gas in fluid communication with the gas inlet port.

7. The surgical device of claim 6, further comprising a regulating mechanism configured to prevent or reduce the flow of gas from the source to the interior cavity.

8. The surgical device of claim 7, wherein the regulating mechanism is selectively operable by a user of the surgical device to allow, to prevent, or to reduce the flow of gas from the source to the interior cavity.

9. The surgical device of claim 3, further comprising a means for supplying a pressurized gas to the gas inlet port.

10. The surgical device of claim 6, wherein the source of pressurized gas is selected from a group consisting of: a pump, a syringe, and a pressurized gas container.

11. The surgical device of claim 2, further comprising a mixer configured for mixing the solution within the interior cavity of the reservoir.

12. The surgical device of claim 11, wherein the mixer comprises a mixing member and a motor, wherein the mixing member is disposed therein the interior cavity of the reservoir, and wherein the motor is coupled to the mixing member such that the solution can be moved within the interior cavity in its cooled state.

13. The surgical device of claim 1, further comprising a cooling pipe configured for maintaining the solution in its cooled state.

14. The surgical device of claim 13, wherein at least a portion of the cooling pipe is positioned within the interior cavity of the reservoir.

15. The surgical device of claim 14, wherein the cooling pipe comprises a coil.

16. The surgical device of claim 13, wherein at least a portion of the cooling pipe is positioned about a portion of an external surface of the reservoir.

17. The surgical device of claim 16, wherein the cooling pipe comprises a coil that is wrapped about and contacts the portion of the external surface of the reservoir.

18. The surgical device of claim 13, wherein at least a portion of the cooling member is embedded within the reservoir.

19. The surgical device of claim 12, wherein the mixing member is configured to crush ice that is disposed therein the interior cavity.

20. The surgical device of claim 19, wherein the mixing member comprises a blade for contacting the ice.

21. The surgical device of claim 19, wherein the mixing member can be selectively activated or inactivated by a user of the surgical device.

22. The surgical device of claim 12, wherein the reservoir has a top portion and a bottom portion, the bottom portion being removeably seatable on a base member, wherein the motor of the mixer is disposed therein an interior portion of the base member.

23. The surgical device of claims 2 or 12, further comprising a means for selectively propelling the solution in its chilled state from the reservoir and out of the second end of the tubular probe.

24. The surgical device of claim 23, wherein the means for propelling comprises an extruding assembly.

25. The surgical device of claim 24, wherein the extruding assembly comprises the mixing body.

26. The surgical device of claim 25, wherein the mixing body comprises a screw configured to rotate about its longitudinal axis.

27. The surgical device of claim 24, wherein the extruding assembly is positioned within the conduit.

28. The surgical device of claim 27, wherein the extruding mechanism comprises a moveable screw configured to rotate about its longitudinal axis.

29. The surgical device of claim 23, wherein the means for propelling comprises pressurized gas located therein the interior cavity.

30. The surgical device of claim 24, further comprising a valve, wherein the valve is positioned between and in fluid communication with the outlet of the reservoir and the first end of the tubular probe.

31. The surgical device of claim 30, wherein the extruding mechanism comprises a screw configured to rotate about its longitudinal axis.

32. The surgical device of claim 23, wherein the means for propelling comprises a pump, wherein the pump is operatively coupled to the conduit.

33. The surgical device of claim 2, wherein the conduit further comprises a regulating assembly configured to prevent or reduce flow of the solution in its chilled state through the conduit.

34. The surgical device of claim 33, wherein at least a portion of the regulating assembly is positioned within the conduit.

35. The surgical device of claim 33, wherein the regulating assembly is coupled to the conduit and comprises means for selectively reducing the size of a lumen of the conduit.

36. The surgical device of claim 1, wherein the probe further comprises a regulating mechanism configured to prevent or reduce flow of the solution in its chilled state through the probe.

37. The surgical device of claim 36, wherein the regulating mechanism is disposed within the lumen of the tubular probe and can block the lumen or a portion thereof.

38. The surgical device of claim 36, wherein the regulating mechanism is located external to the lumen of the tubular probe and can operatively reduce the lumen diameter.

39. The surgical device of claims 33 or 36, wherein the regulating mechanism is selectively operable by a user of the surgical device to allow, to prevent, or to reduce flow of the cooled solution through the tube lumen.

40. The surgical device of claim 1, wherein an insulating sleeve is positioned about at least a portion of the external surface of the reservoir.

41. The surgical device of claim 1, wherein the reservoir is configured to insulate the solution located therein the interior cavity.

42. A surgical device, comprising:
a reservoir defining an interior cavity and having a gas inlet port configured for the introduction of gas into the interior cavity of the reservoir to pressurize the interior cavity of the reservoir with the gas, wherein the reservoir is configured to maintain a solution disposed therein the interior cavity in a cooled state; and
a tubular probe having a first end and an opposed second end, and wherein the first end of the tubular probe is in selective fluid communication with the interior cavity of the reservoir such that the solution in the cooled state can be selectively dispensed from the second end of the tubular probe into a subject.

43. The surgical device of claim 42, wherein the reservoir further comprises a gas outlet port configured for release of gas from the interior cavity of the reservoir.

44. The surgical device of claim 42, further comprising a mixer configured for mixing the solution within the interior cavity of the reservoir.

45. The surgical device of claim 44, wherein the mixer comprises a mixing member and a motor, wherein the mixing member is disposed therein the interior cavity of the reservoir, and wherein the motor is coupled to the mixing member such that the solution can be moved within the interior cavity in its cooled state.

46. The surgical device of claim 45, further comprising a regulating mechanism configured to prevent or reduce the flow of gas from the source to the interior cavity.

47. The surgical device of claim 46, wherein the regulating mechanism is selectively operable by a user of the surgical device to allow, to prevent or to reduce the flow of gas from the source to the interior cavity.

48. The surgical device of claim 47, wherein the mixing member can be selectively activated or inactivated by a user of the surgical device.
